SEQUENCE LISTING (23 sequences)

SEQ ID NO: 1 - DNA, 18 bp, Artificial Sequence
  Description of Artificial Sequence oligonucleotide primers
gtcctggaggagcccaac

SEQ ID NO: 2 - DNA, 18 bp, Artificial Sequence
  Description of Artificial Sequence oligonucleotide primers
gcagtcctgctcaggctc

SEQ ID NO: 3 - DNA, 21 bp, Artificial Sequence
  Description of Artificial Sequence oligonucleotide primers
gtgggtgctaagggtaaggga

SEQ ID NO: 4 - DNA, 21 bp, Artificial Sequence
  Description of Artificial Sequence oligonucleotide primers
tgggattatggggtggtggca

SEQ ID NO: 5 - DNA, 21 bp, Artificial Sequence
  Description of Artificial Sequence oligonucleotide primers
cagtggatcatggacctcttc

SEQ ID NO: 6 - DNA, 20 bp, Artificial Sequence
  Description of Artificial Sequence oligonucleotide primers
gcgtagtggtgttccggctt

SEQ ID NO: 7 - DNA, 21 bp, Artificial Sequence
  Description of Artificial Sequence oligonucleotide primers
gctgaccaacacaaagacggg

SEQ ID NO: 8 - DNA, 22 bp, Artificial Sequence
  Description of Artificial Sequence oligonucleotide primers
ataaccaccataaggaccgagg

SEQ ID NO: 9 - DNA, 21 bp, Artificial Sequence
  Description of Artificial Sequence oligonucleotide primers
atgaagaccccgctgtaccct

SEQ ID NO: 10 - DNA, 22 bp, Artificial Sequence
  Description of Artificial Sequence oligonucleotide primers
tctcgtgtgttttctcttcccg

SEQ ID NO: 11 - DNA, 20 bp, Artificial Sequence
  Description of Artificial Sequence oligonucleotide primers
gcagcatcggcttcagcaag

SEQ ID NO: 12 - DNA, 19 bp, Artificial Sequence
  Description of Artificial Sequence oligonucleotide primers
ctagcaggtccgcaaggtg

SEQ ID NO: 13 - DNA, 21 bp, Artificial Sequence
  Description of Artificial Sequence oligonucleotide primers
gcctttgtgaaccaacacctg

SEQ ID NO: 14 - DNA, 21 bp, Artificial Sequence
  Description of Artificial Sequence oligonucleotide primers
gttgcagtagttctccagctg

SEQ ID NO: 15 - DNA, 20 bp, Artificial Sequence
  Description of Artificial Sequence oligonucleotide primers
gaattcattgcttggctggt

SEQ ID NO: 16 - DNA, 20 bp, Artificial Sequence
  Description of Artificial Sequence oligonucleotide primers
catttcaaacatcccacgtg

SEQ ID NO: 17 - DNA, 21 bp, Artificial Sequence
  Description of Artificial Sequence oligonucleotide primers
cgtcagtttctgcagaagtcc

SEQ ID NO: 18 - DNA, 20 bp, Artificial Sequence
  Description of Artificial Sequence oligonucleotide primers
ccatagccgggtttgagtta

SEQ ID NO: 19 - DNA, 21 bp, Artificial Sequence
  Description of Artificial Sequence oligonucleotide primers
gctgccgcacgcctctgcctc

SEQ ID NO: 20 - DNA, 21 bp, Artificial Sequence
  Description of Artificial Sequence oligonucleotide primers
aagtccagcgggctgagctcc

SEQ ID NO: 21 - DNA, 20 bp, Artificial Sequence
  Description of Artificial Sequence oligonucleotide primers
cactttccaccagtcccata

SEQ ID NO: 22 - DNA, 21 bp, Artificial Sequence
  Description of Artificial Sequence oligonucleotide primers
gtttacttcctccagggaaac

SEQ ID NO: 23 - DNA, 18 bp, Artificial Sequence
  Description of Artificial Sequence oligonucleotide primers
aatccgtcgagcagagtt

(19) United States
(12) Patent Application Publication
(10) Pub. No.: US 2004/0005301 A1
(43) Pub. Date: Jan. 8, 2004

(54) Identification and high-yield isolation of human pancreatic islet progenitor and stem cells

(76) Inventors: Steven A. Goldman, South Salem, NY (US); Hansoo Michael Keyoung, New York, NY (US)

Correspondence Address: Michael L. Goldman, NIXON PEABODY LLP, Clinton Square, P.O. Box 31051, Rochester, NY, 14603-1051, US

(21) Appl. No.: 10/364,029

(22) Filed: Feb. 10, 2003

Related U.S. Application Data
(60) Provisional application No. 60/356,556, filed on Feb. 12, 2002.

(51) Int. Cl. A61K 48/00; C12Q 1/00; C12N 5/08

(52) U.S. Cl. 424/93.21; 435/366; 435/4

(57) ABSTRACT

The present invention relates to a method of separating pancreatic Islet cells or progenitor or stem cells thereof from a mixed population of cells from the pancreas. This involves selecting an enhancer/promoter which functions in the pancreatic Islet cells or progenitor cells thereof and introducing a nucleic acid molecule encoding a fluorescent protein under control of the enhancer/promoter into the mixed population of cells. The pancreatic Islet cells or progenitor or stem cells thereof are then allowed to express the fluorescent protein. The fluorescent cells (i.e. the said pancreatic Islet cells or progenitor or stem cells thereof) are then separated from the mixed population of cells. Also disclosed is an enriched or purified preparation of isolated human pancreatic Islet cells or progenitor or stem cells and the use of these cells in a method of treating a diabetic condition by transplanting the cells into a subject.

This application claims benefit of U.S. Provisional Patent Application Serial No. 60/356,556, filed Feb. 12, 2002.

### FIELD OF THE INVENTION

The present invention relates to a method of separating pancreatic Islet cells or progenitor or stem cells thereof, the resulting separated cells, and their use in treating a diabetic condition.

### BACKGROUND OF THE INVENTION

Beta cells of the human pancreas have been envisioned as a potential cellular vector for cell-based therapy of type 1 diabetes, in which beta cells are specifically lost. However, these cells have not been amenable to therapeutic use, since they are a lineage-restricted phenotype capable of limited expansion once isolated. This limitation has led to efforts to raise Islet cell progenitors, capable of both expansion and beta cell production. However, these studies have hitherto been limited by the lack of any effective means of prospectively identifying Islet progenitor cells, which, as a result, have never been selectively isolated or purified as such. To circumvent this problem, current strategies for beta cell replacement have focused on the transplantation of mechanically-sorted whole Islets extracted from cadaveric adult pancreas (Calafiore R. “Perspectives in Pancreatic and Islet Cell Transplantation for the Therapy of IDDM,” Diabetes Care 20(5):889-96 (1997); Soria et al. “From Stem cells to Beta Cells: New Strategies in Cell Therapy of Diabetes Mellitus,” Diabetologia 44(4):407-15 (2001)). However, these strategies are limited by the scarcity of appropriate cadaveric samples, the inconsistent tissue and cell quality of these samples, the need to use these fully differentiated β cells soon after harvest, the very limited capability of adult pancreatic neuroendocrine cells for mitotic expansion, and the impure and heterogenous nature of mechanically-sorted Islets, which include stromal and endothelial cells and attached acinar cells, as well as neuroendrocine cells.

To address these deficiencies, applicants have developed the present invention as a means of isolating pancreatic Islet cells or progenitor or stem cells thereof, from both fetal- and adult-derived pancreas, which can then be mitotically expanded for a prolonged period and instructively differentiated to insulin-secreting β cells when derived. These in vitro-generated human β cells can then be used for the cell-based treatment of diabetes.

### SUMMARY OF THE INVENTION

The present invention relates to a method of separating pancreatic Islet cells or progenitor or stem cells thereof from a mixed population of cells from the pancreas. This involves selecting an enhancer/promoter which functions in the pancreatic Islet cells or progenitor cells thereof and introducing a nucleic acid molecule encoding a fluorescent protein under control of the enhancer/promoter into the mixed population of cells. The pancreatic Islet cells or progenitor or stem cells thereof are then allowed to express the fluorescent protein. The fluorescent cells (i.e. the said pancreatic Islet cells or progenitor or stem cells thereof) are then separated from the mixed population of cells.

Another aspect of the present invention relates to an enriched or purified preparation of isolated human pancreatic Islet cells or progenitor or stem cells.

A further aspect of the present invention relates to a method of treating a diabetic condition. This involves providing an enriched or purified preparation of pancreatic Islet progenitor or stem cells or Islet cells differentiated therefrom and transplanting the cells into a subject under conditions effective to treat a diabetic condition.

The advent of promoter-specified, green fluorescent protein (GFP)-based cell sorting, which separates cells based on their transcriptional activity rather than surface marker expression has led to the isolation and purification of a variety of otherwise scarce phenotypes (Keyoung et al. “High-Yield Selection and Extraction of Two Promoter-Defined Phenotypes of Neural Stem Cells From the Fetal Human Brain,” Nat Biotechnol 19(9):843-50 (2001); Komblum et al. “Molecular Markers in CNS Stem Cell Research: Hitting a Moving Target,” Nat Rev Neurosci 2(11):843-6 (2001), which are hereby incorporated by reference in their entirety). This strategy has proven of particular use in isolating neural progenitor cells of a variety of phenotypes from the central nervous system. Yet developing pancreatic Islets, which may be of neuroendocrine ontogeny, appear to express many of the same developmental genes as do brain cells (Kim et al. “Intercellular Signals Regulating Pancreas Development and Function,” Genes Dev 15(2):111-27 (2001), which is hereby incorporated by reference in its entirety). A number of transcription factors typically associated with neural ontogeny, including Nkx6.1, the neurogenin family, Islet 1, and Hb9, are expressed likewise during the ontogeny of pancreatic Islets (Gu et al. “Direct Evidence for the Pancreatic Lineage: NGN3+ Cells are Islet Progenitors and are Distinct From Duct Progenitors,” Development 129(10):2447-57 (2002); Jensen et al. “Independent Development of Pancreatic Alpha- and Beta-Cells From Neurogenin3-Expressing Precursors: a Role for the Notch Pathway in Repression of Premature Differentiation,” Diabetes 49(2):163-76 (2000); Schwitzgebel et al. “Expression of Neurogenin3 Reveals an Islet Cell Precursor Population in the Pancreas,” Development 127(16):3533-42 (2000), which are hereby incorporated by reference in their entirety). More broadly, several recent studies have identified an early neural filament protein, nestin, in developing Islets, as well as in scattered cells within the adult Islets (Zhang et al. “Up-Regulation of the Expression of Activins in the Pancreatic Duct by Reduction of the Beta-Cell Mass,” Endocrinology 143(9):3540-7 (2002); Zulewski et al. “Multipotential Nestin-Positive Stem Cells Isolated From Adult Pancreatic Islets Differentiate ex vivo Into Pancreatic Endocrine, Exocrine, and Hepatic Phenotypes,” Diabetes 50(3):521-33 (2001); Hunziker et al. “Nestin-Expressing Cells in the Pancreatic Islets of Langerhans,” Biochem Biophys Res Commun 271(1):116-9 (2000), which are hereby incorporated by reference in their entirety). Indeed, both pancreatic stem cells and Islet progenitors express nestin protein (Hunziker et al. “Nestin-Expressing Cells in the Pancreatic Islets of Langerhans,” Biochem Biophys Res Commun 271(1):116-9 (2000) and Zulewski et al. “Multipotential Nestin-Positive Stem Cells Isolated From Adult Pancreatic Islets Differentiate ex vivo Into Pancreatic Endocrine, Exocrine, and Hepatic Phenotypes,” Diabetes 50(3):521-33 (2001), which are hereby incorporated by reference in their entirety). On this basis, applicants reasoned that if Islet progenitors expressed nestin under the control of the same regulatory region—the second intronic enhancer of the nestin gene (Zimmerman et al., “Independent Regulatory Elements in the Nestin Gene Direct Transgene Expression to Neural Stem Cells or Muscle Precursors,” Neuron 12(1):11-24 (1994), which is hereby incorporated by reference in its entirety) as did neural stem cells, then E/nestin:GFP-based sorting of the fetal pancreas might be used to isolate the human pancreatic Islet progenitor cell.

The examples of the present invention show that by transducing dissociates of the human fetal pancreas with adenoviruses encoding GFP, placed under nestin regulatory control, Islet progenitor cells may indeed be specifically identified and then extracted by fluorescence-activated cell sorting. These nestin:GFP-sorted pancreatic progenitors are mitotically competent and expand in bFGF to form Islets in vitro. They then differentiate in activin A to become the insulin, glucagons, and somatostatin-secreting cells typical of mature Islets. The beta cells thereby generated secrete insulin, and respond appropriately to glucose exposure by increasing their secretion of insulin. The Islet cell cultures thus achieved are essentially pure, and the levels of insulin secreted by their beta cells are higher than any yet reported by beta cells generated from any cell type in vitro. This approach may therefore permit human Islet progenitor cells and their beta cell progeny to be prepared in the purity and quantity needed for cell-based therapeutic strategies.

### BRIEF DESCRIPTION OF THE DRAWINGS

FIG. 1 is a schematic drawing summarizing the method by which Islet stem and progenitor cells are isolated from the pancreas in accordance with the present invention.

FIGS. 2A-G show human fetal pancreas tissue sections immunostained for nestin protein and endocrine markers. FIG. 2A shows human fetal pancreas 23 week cryosections stained for nestin and endocrine markers of glucagons, somatostatin, and insulin. There was no visible co-labeling of nestin and glucagons (FIGS. 2B and 2C) and nestin and somatostatin (FIGS. 2E and 2F). FIG. 2G shows that the pancreatic Islet does not contain nestin cells but does co-express insulin. The z-dimension reconstruction was observed orthogonally in both xz and yz planes; this is shown below and to the left of the z-dimension composite. Scale bars=10 μm.

FIGS. 3A-H show the live labeling of E.nestin:EGFP cells from human pancreas and FACS isolation of EGFP-expressing nestin cells. FIGS. 3A and 3D show fresh dissociates from human pancreas tissue infected with E/nestin.EGFP express E/nestin by 4 DIV. FIGS. 3B, 3C, 3E, and 3F show Islet-like clusters contain cells that express E/Nestin.EGFP when cultured in suspension (FIGS. 3B and 3E) or on matrigel (FIGS. 3C and 3F). These pancreatic cells were re-dissociated to single cells and then sorted on the basis of AdP/CMV:lacZ (FIG. 3G; a nonfluorescent control), or AdE/nestin:EGFP (FIG. 3H). The GFP fluorescence intensity (FL1) was plotted against cell size (forward scatter, FSC). Approximately 3.6% of the AdE/nestin:EGFP-infected cells achieved arbitrary threshold fluorescence intensity; using the same acceptance criteria, only 0.05% of cells infected with nonfluorescent AdP/CMV:lacZ were recognized.

FIG. 4 shows a TRAP assay. AdE/nestin.EGFP-positive sorted cells and negatively selected AdE/nestin-negative cells were FACS isolated and collected for Telomerase Repeat Amplification Protocol assay (TRAP). Immediately post-FACS isolation, AdE/nestin.EGFP-positive sorted and AdE/nestin-negative cells were collected and telomerase from each samples were extracted. Odd lanes contain cells at different densities that underwent TRAP assay for both cell populations. Lane 1 is a positive primer template control at concentration of 0.2M. Even lanes contain denatured telomerase that were heat-inactivated to serve as controls. The lane that contains 5,000 AdE/nestin.EGFP-positive cells (lane 5) exhibited positive telomerase activity and distinct ladders demonstrating telomerase activity can be seen with 10,000 AdE/nestin.EGFP-positive cells (lane 7). AdE/nestin.EGFP-negative cells did not exhibit any telomerase activity when equal cell number of cells were assayed compared to the AdE/nestin.EGFP-positive cells (lanes 8-13).

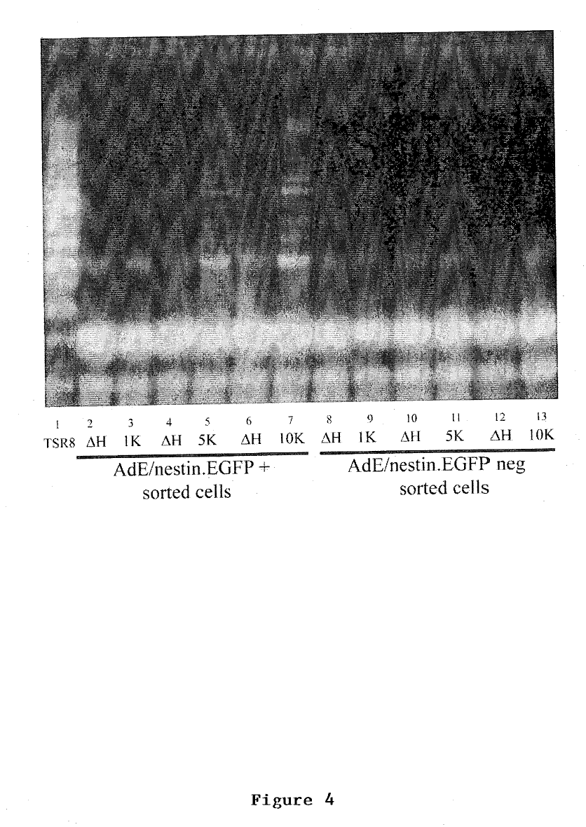

FIGS. 5A-B show the transcriptional profile via RT-PCR of E/Nestin:EGFP cells at different culture conditions. As shown in FIG. 5A, under proliferating conditions, immediately after FACS isolation, E/nestin-sorted cells expressed HNFB, pdx-1, Ngn3, Nkx6.1, Nkx2.2, and Islet1 but lacked expression for differentiated endocrine markers such as insulin, glucagon, somatostatin, and pancreatic polypeptide. As shown in FIG. 5B, after in vitro differentiation in activin containing high-glucose culture condition, nestin-sorted cells continued to express Islet1 in addition to known endocrine markers such as insulin, glucagon, somatostatin and pancreatic polypeptide.

FIGS. 6A-D show the E/nestin:EGFP FACS-sorted cells differentiate into endocrine cells. When E/nestin.GFP-sorted cells were cultured under Activin-A containing conditions for 7-9 days differentiated into (FIG. 6A) insulin-expressing cells, (FIG. 6B) glucagon-expressing cells, (FIG. 6C) somatostatin-expressing cells, and (FIG. 6D) pancreatic polypeptide-expressing cells. After 4 months in vitro, Islet-like clusters formed and became insulin-positive cells after prolonged expansion ex vivo. Some of the post-sorted cells retained nestin-protein expression.

FIGS. 7A-C show enrichment of insulin-producing cells via FACS isolation for E/nestin.EGFP-positive cells and then differentiation with Activin A. FIG. 7A shows the percentage of insulin-positive cells cultured in bFGF comparing the E/nestin-sorted cells (n=3) versus unsorted cells (n=4). FIG. 7B shows the percentage of insulin-positive cells cultured in activin when compared E/nestin.EGPF-sorted cells (n=3) to unsorted cells (n=3) after FACS isolation. The E/nestin.EGFP cells exhibited almost 44-fold increase in differentiation to insulin-expressing cells in response to activin. FIG. 7C shows that compared to unsorted cells, coupling FACS-isolation with Activin induction, the E/nestin-EGFP-sorted cells yielded a higher percentage of insulin-positive cells than the percentage found immediately post-tissue dissociation. This increase attributed to FACS isolation for E/nestin cells (n=3) was almost a five-fold increase in insulin-positive cells over the unsorted cells (n=3); dark-colored bars, unsorted; light-colored bars, E/nestin.EGFP-sorted cells; *p<0.05.

### DETAILED DESCRIPTION OF THE INVENTION

As used herein, the term “isolated” when used in conjunction with a nucleic acid molecule refers to: 1) a nucleic acid molecule which has been separated from an organism in a substantially purified form (i.e. substantially free of other substances originating from that organism), or 2) a nucleic acid molecule having the same nucleotide sequence but not necessarily separated from the organism (i.e. synthesized or recombinantly produced nucleic acid molecules).

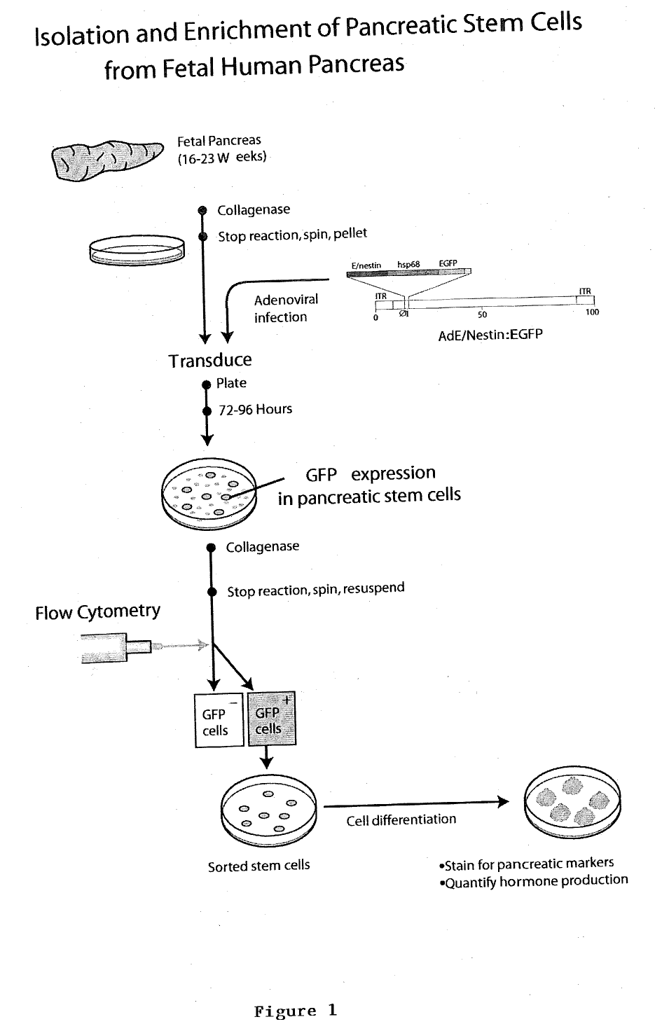

The present invention relates to a method of separating pancreatic Islet cells or progenitor or stem cells thereof from a mixed population of cells from the pancreas. This involves selecting an enhancer/promoter which functions in the pancreatic Islet cells or progenitor cells thereof and introducing a nucleic acid molecule encoding a fluorescent protein under control of the enhancer/promoter into the mixed population of cells. The pancreatic Islet cells or progenitor or stem cells thereof are then allowed to express the fluorescent protein. The fluorescent cells (i.e. the said pancreatic Islet cells or progenitor or stem cells thereof) are then separated from the mixed population of cells. The process of selecting progenitors to select a particular cell type from a mixed population of cell types using a promoter that functions in the progenitor cells and a nucleic acid encoding a marker protein is described in U.S. Pat. No. 6,245,564 to Goldman et. al., which is hereby incorporated by reference in its entirety. See FIG. 1.

The cells of particular interest according to the present invention are pancreatic Islet cells or progenitor cells or stem cells thereof. Any of these cells which one desires to separate from a plurality of cells and immortalize can be selected in accordance with the present invention, as long as a promoter specific for the chosen cell is available. “Specific” , as used herein to describe a promoter, means that the promoter functions only in the chosen cell type. A chosen cell type can refer to different types of cells or different stages in the developmental cycle of a progenitor cell. For example, the chosen cell may be committed to a particular adult cell phenotype and the chosen promoter only functions in that progenitor cell; i.e. the promoter does not function in adult cells. Although committed and uncommitted progenitor cells may both be considered progenitor cells, these cells are at different stages of progenitor cell development and can be separated according to the present invention if the chosen promoter is specific to the particular stage of the progenitor cell. Those of ordinary skill in the art can readily determine a cell of interest to select based on the availability of a promoter specific for that cell of interest.

The promoter suitable for carrying out this aspect of the present invention can be the E/nestin enhancer/promoter, the Musashi promoter, the NKX6.1 promoter, the neurogenin-3 promoter, the HB9 promoter, or the PDX-1 promoter.

Having determined the cell of interest and selected a promoter specific for the cell of interest, a nucleic acid molecule encoding a protein marker, preferably a green fluorescent protein, under the control of the promoter is introduced into a plurality of cells to be sorted.

The isolated nucleic acid molecule encoding a green fluorescent protein can be deoxyribonucleic acid (DNA) or ribonucleic acid (RNA, including messenger RNA or mRNA), genomic or recombinant, biologically isolated or synthetic. The DNA molecule can be a cDNA molecule, which is a DNA copy of a messenger RNA (mRNA) encoding the GFP. In one embodiment, the GFP can be from Aequorea victoria (U.S. Pat. No. 5,491,084 to Prasher et. al.,). A plasmid designated pGFP10.1 has been deposited pursuant to, and in satisfaction of, the requirements of the Budapest Treaty on the International Recognition of the Deposit of Microorganisms for the Purposes of Patent Procedure, with the American Type Culture Collection (ATCC), 12301 Parklawn Drive, Rockville, Md. 20852 under ATCC Accession No. 75547 on Sep. 1, 1993. This plasmid is commercially available from the ATCC due to the issuance of U.S. Pat. No. 5,491,084 on Feb. 13, 1996 in which the plasmid is described. This plasmid comprises a cDNA which encodes a green fluorescent protein (GFP) of Aequorea victoria as disclosed in U.S. Pat. No. 5,491,084 to Chalfie et al., the contents of which are incorporated herein by reference in its entirety. A mutated form of this GFP (a red-shifted mutant form) designated pRSGFP-C1 is commercially available from Clontech Laboratories, Inc. (Palo Alto, Calif.).

The plasmid designated pTα1-RSGFP has been deposited pursuant to, and in satisfaction of, the requirements of the Budapest Treaty on the International Recognition of the Deposit of Microorganisms for the Purposes of Patent Procedure, with the American Type Culture Collection (ATCC), 12301 Parklawn Drive, Rockville, Md. 20852 under ATCC Accession No. 98298 on Jan. 21, 1997. This plasmid uses the red shifted GFP (RS-GFP) of Clontech Laboratories, Inc. (Palo Alto, Calif.), and the Tα1 promoter sequence provided by Dr. F. Miller (Montreal Neurological Institute, McGill University, Montreal, Canada). In accordance with the subject invention, the Tα1 promoter can be replaced with another specific promoter, and the RS-GFP gene can be replaced with another form of GFP, by using standard restriction enzymes and ligation procedures.

Mutated forms of GFP that emit more strongly than the native protein, as well as forms of GFP amenable to stable translation in higher vertebrates, are now available and can be used for the same purpose. The plasmid designated pTα1-GFPh has been deposited pursuant to, and in satisfaction of, the requirements of the Budapest Treaty on the International Recognition of the Deposit of Microorganisms for the Purposes of Patent Procedure, with the American Type Culture Collection (ATCC), 12301 Parklawn Drive, Rockville, Md. 20852 under ATCC Accession No. 98299 on Jan. 21, 1997. This plasmid uses the humanized GFP (GFPh) of Zolotukhin and Muzyczka (Levy et al., Nature Biotechnol. 14:610-14 (1996), which is hereby incorporated by reference in its entirety) and the Tα1 promoter sequence provided by Dr. F. Miller (Montreal). In accordance with the subject invention, the Tα1 promoter can be replaced with another specific promoter, and the GFPh gene can be replaced with another form of GFP, by using standard restriction enzymes and ligation procedures. Any nucleic acid molecule encoding a fluorescent form of GFP can be used in accordance with the subject invention.

Standard techniques are then used to place the nucleic acid molecule encoding GFP under the control of the chosen cell specific promoter. Generally, this involves the use of restriction enzymes and ligation.

The resulting construct, which comprises the nucleic acid molecule encoding the GFP under the control of the selected promoter (itself a nucleic acid molecule) (with other suitable regulatory elements if desired), is then introduced into a plurality of cells which are to be sorted. Techniques for introducing the nucleic acid molecules of the construct into the plurality of cells may involve the use of expression vectors which comprise the nucleic acid molecules. These expression vectors (such as plasmids and viruses) can then be used to introduce the nucleic acid molecules into the plurality of cells.

Various methods are known in the art for introducing nucleic acid molecules into host cells. These include: 1) microinjection, in which DNA is injected directly into the nucleus of cells through fine glass needles; 2) dextran incubation, in which DNA is incubated with an inert carbohydrate polymer (dextran) to which a positively charged chemical group (DEAE, for diethylaminoethyl) has been coupled (the DNA sticks to the DEAE-dextran via its negatively charged phosphate groups, large DNA-containing particles stick in turn to the surfaces of cells (which are thought to take them in by a process known as endocytosis), and some of the DNA evades destruction in the cytoplasm of the cell and escapes to the nucleus, where it can be transcribed into RNA like any other gene in the cell); 3) calcium phosphate coprecipitation, in which cells efficiently take in DNA in the form of a precipitate with calcium phosphate; 4) electroporation, in which cells are placed in a solution containing DNA and subjected to a brief electrical pulse that causes holes to open transiently in their membranes so that DNA enters through the holes directly into the cytoplasm, bypassing the endocytotic vesicles through which they pass in the DEAE-dextran and calcium phosphate procedures (passage through these vesicles may sometimes destroy or damage DNA); 5) liposomal mediated transformation, in which DNA is incorporated into artificial lipid vesicles, liposomes, which fuse with the cell membrane, delivering their contents directly into the cytoplasm; 6) biolistic transformation, in which DNA is absorbed to the surface of gold particles and fired into cells under high pressure using a ballistic device; 7) naked DNA insertion; and 8) viral-mediated transformation, in which nucleic acid molecules are introduced into cells using viral vectors. Since viral growth depends on the ability to get the viral genome into cells, viruses have devised efficient methods for doing so. These viruses include retroviruses, lentivirus, adenovirus, herpesvirus, and adeno-associated virus.

As indicated, some of these methods of transforming a cell require the use of an intermediate plasmid vector. U.S. Pat. No. 4,237,224 to Cohen and Boyer, which is hereby incorporated by reference in its entirety, describes the production of expression systems in the form of recombinant plasmids using restriction enzyme cleavage and ligation with DNA ligase. These recombinant plasmids are then introduced by means of transformation and replicated in unicellular cultures including procaryotic organisms and eucaryotic cells grown in tissue culture. The DNA sequences are cloned into the plasmid vector using standard cloning procedures known in the art, as described by Sambrook et al. Molecular Cloning: A Laboratory Manual, 2d Edition, Cold Spring Harbor Laboratory Press, Cold Spring Harbor, N.Y. (1989), which is hereby incorporated by reference in its entirety.

In accordance with one of the above-described methods, the nucleic acid molecule encoding the GFP is thus introduced into a plurality of cells. The promoter which controls expression of the GFP, however, only functions in the cell of interest. Therefore, the GFP is only expressed in the cell of interest. Since GFP is a fluorescent protein, the cells of interest can therefore be identified from among the plurality of cells by the fluorescence of the GFP.

Any suitable means of detecting the fluorescent cells can be used. The cells may be identified using epifluorescence optics, and can be physically picked up and brought together by Laser Tweezers (Cell Robotics Inc., Albuquerque, N. Mex.). They can be separated in bulk through fluorescence activated cell sorting, a method that effectively separates the fluorescent cells from the non-fluorescent cells.

The fluorescent oligodendrocyte progenitor cells are then separated from the mixed population of cells by fluorescence activated cell sorting.

The mixed population of cells is derived from pancreatic tissue or a pancreatic cell culture. The cells preferably come from a human including an adult or a fetus.

Another aspect of the present invention relates to an enriched or purified preparation of isolated human pancreatic Islet cells or progenitor or stem cells thereof The enriched or purified preparation of isolated human pancreatic Islet cells or progenitor or stem cells thereof is at least 90% pure, preferably at least 95% pure, and most preferably at least 99% pure.

A further aspect of the present invention relates to a method of treating a diabetic condition. This involves providing an enriched or purified preparation of pancreatic Islet progenitor or stem cells or Islet cells differentiated therefrom and transplanting the cells into a subject under conditions effective to treat a diabetic condition.

The diabetic condition treated in accordance with this aspect of the present invention can be Diabetes Mellitus Type 1 or Diabetes Mellitus Type 2.

Transplantation of the enriched or purified population is carried out by injecting 1×10⁵ to 1×10⁷ cells into the diabetic host subject. These injections may be delivered subcutaneously or into the residual pancreas, or, alternatively, into the renal subeapsular space, or other suitable host locations. Following transplantation, the subject may be immunosuppressed to accommodate incomplete cross-matching as necessary, using standard chronic immunosuppressive therapy, including, but not limited to, glucocorticoids, cyclosporin, cyclophosphamide, and FK506.

Desirably, the transplanted cells produce insulin and/or glucagon.

### EXAMPLES

### Example 1

Human Fetal Culture

Human fetal pancreas (HFP) was procured at hospitals or from Advance Bioscience Research, Inc. (Alameda, Calif.) (ABR). These HFP samples (n=25) were collected into Ca/Mg-free HBSS, then dissected. The cut pieces were incubated in 0.8 mg/ml of Collagenase P with gentle shaking for 15 minutes at 37° C. Following incubation, the tissue was collected by centrifuging at 200g for 5 min in an IEC Centra-4B centrifuge, resuspended in Modified RPMI 1640 with DNase 1(10 units/ml) and incubated for 10 min at 37° C. The samples were spun and the pellets resuspended in 2 ml of media, then dissociated by sequentially triturating for 20, 10, and 5 times, through three serially-narrowed glass Pasteur pipettes. The dissociated cells were purified by passing through a 40 μm Cell Strainer (Becton Dickinson), rinsed with RPMI 1640 containing 10% characterized fetal bovine serum, FBS) and resuspended at 1×10⁶ cells/ml in in Modified DMEM/F12/N2 supplemented with 0.1 mM Nonessential Amino-acid, 3.8 mM Hepes buffer, 0.01 % BSA, 1 mM Pyruvate, 2 mM of Glutamine containing 20 ng/ml bFGF(Sigma), and 10 ng/ml EGF (Sigma). The cells were plated at 1 ml/dish into 6 well low-cluster suspension plates, and incubated at 37° C. in 5% CO₂. For some cultures, 30 μM bromodeoxyuridine (BrdU 36), was added to the medium in order to label dividing cells.

### Example 2

Flow Cytometry and Sorting

Flow cytometry and sorting of hGFP+ cells was performed on a FACS Vantage (Becton-Dickinson). Cells were washed twice with Ca⁺⁺, Mg⁺⁺-free Hanks' Balanced Salt Solution (HBSS), then dissociated by incubation in non-enzymatic dissociation protocol (Sigma) for 5 min at 37° C. The dissociation reaction was terminated by PRMI 1640 containing 10% FBS. The cells (1×10⁶/ml) were analyzed by light forward and right-angle (side) scatter, and for GFP fluorescence through a 510±20 nm bandpass filter, as they traversed the beam of a Coherent INNOVA Enterprise II Ion Laser (488 nm, 100 mW). Sorting was done using a purification-mode algorithm. The E/nestin:lacZ transfected cells were used as a control to set the background fluorescence; a false positive rate of 0.1-0.3% was accepted to ensure an adequate yield. For those samples transfected with E/nestin:EGFP, cells detected as be more fluorescent than background were sorted at 1000-3000 cells/sec.

### Example 3

Immunostaining and Imaging

In vivo

Human pancreas tissue was fixed in 4% paraformaldehyde. The tissue was frozen, mounted in OCT cryomounting media (Lipshaw), and cryosectioned at 15 μm. The sections were washed with PBS, perneabilized with PBS/0.1% saponin/1% NGS for 20 min at RT, and blocked with PBS/0.05% saponin/5% NGS for 20 min at RT. Sections were then labeled with either: mouse anti-Insulin, 1:100 (Chemicon), mouse anti-glucagon, 1:2000 (Sigma), rat anti-somatostatin, 1:100 (Chemicon), rabbit anti-PP, 1:100 (Chemicon), mouse anti-cytokeratin-19, (DAKO) , rabbit anti-Amylase, 1:100 (Biomeda), mouse anti-synaptophysin, 1:1000 (Chemicon), or nestin (rabbit anti-human nestin, 1:1000; courtesy of U. Lendahl). Fluorescent-labeled secondary antibodies were then used at a dilution of 1:100, for 1 hr at RT. To counterstain cell nuclei, fixed cultures were incubated for 2 min in 4′6-diamidino-2-phenylindole dihydrochloride (DAPI, 10 μg/ml Molecular Probes, Eugene, Oreg.).

In vitro

After 14 DIV in differentiation conditions, the cultures were fixed for immunocytochemistry. They were first rinsed with Hanks' balanced salt solution (HBSS), then fixed with 4% paraformaldehyde for 5 min at room temperature. The plates were then stained for either insulin, glucagon, somatostatin, PP, amylase, synaptophysin, and nestin. Incubation in rat anti-BrdU (1:200, Harlan) was performed overnight at 4° C., and secondary antibodies were then used as noted. DAPI was then used as above to counterstain cultured cells.

Cell Counts

The immunostained cultures were observed by immunofluorescence using an Olympus IX70. For each of the plates, the numbers of immunostained cells were counted and scored as a fraction of the total number of cells within that field. For each phenotype assessed, at least three samples were scored, each over at least 10 randomly chosen fields that included a minimum of 200 total cells.

### Example 4

RNA Preparation RT-PCR

The E/nestin.EGFP-sorted human pancreatic cell RNA was extracted immediately post-FACS. The cells were washed in Ca-and Mg-free HBSS, harvested by mild trypsinization (0.25% trypsin, 5 min 37° C.), stopped by the addition of soybean trypsin inhibitor (1 mg/ml), and centrifuged in an IEC clinical centrifuge for 10 min to yield a pellet. The pellet was quickly frozen in liquid nitrogen and then stored at −70° C. Total cellular RNA was first extracted using RNAeasy(Quagen), according to manufacturer's protocol. The yield and purity were estimated by spectrophotometric measurement of OD260/280 ratio.

One microgram of cellular RNA was treated with DNAase (5 U/μl; Pharmacia), in the presence of 5× First strand Buffer (Gibco), 0.1M dithiothreitol (Biorad), RNA Guard RNase inhibitor (40 U/μl; Pharmacia), and DEPC water (Ambion), for 60 min at 37° C. Samples were then subjected to reverse transcription in the presence of 10 mM dNTP (Phamacia), 2.5 μM oligo (dT) (Perkin Elmer), and 200 U/μl MLV reverse transcriptase (Gibco), for 60 min at 42° C. Three microliters of cDNA was then amplified through PCR, using primers specific for transcription factors: hPDX-1, hNgn3, hInsulin, hGlucagon, hSomatostain, hPancreatic Polypeptide, hIslet-1, hNkx6.1, hNKX2.2, hHNF-3β (Table 1).

TABLE 1

| | | |
|---|---|---|
| pdx1 | 5′-GTCCTGGAGGAGCCCAAC-3′ | (SEQ ID NO:1) |
| | 3′-GCAGTCCTGCTCAGGCTC-3′ | (SEQ ID NO:2) |
| ngn3 | 5′-GTGGGTGCTAAGGGTAAGGGA-3′ | (SEQ ID NO:3) |
| | 3′-TGGGATTATGGGGTGGTGGCA-5′ | (SEQ ID NO:4) |
| HNF3beta (Foxa2) | 5′-CAGTGGATCATGGACCTCTTC-3′ | (SEQ ID NO:5) |
| | 3′-GCGTAGTGGTGTTCCGGCTT-5′ | (SEQ ID NO:6) |
| nkx2.2 | 5′-GCTGACCAACACAAAGACGGG-3′ | (SEQ ID NO:7) |
| | 3′-ATAACCACCATAAGGACCGAGG-5′ | (SEQ ID NO:8) |
| nkx6.1 | 5′-ATGAAGACCCCGCTGTACCCT-3′ | (SEQ ID NO:9) |
| | 3′-TCTCGTGTGTTTTCTCTTCCCG-5′ | (SEQ ID NO:10) |
| islet1 | 5′-GCAGCATCGGCTTCAGCAAG-3′ | (SEQ ID NO:11) |
| | 3′-CTAGCAGGTCCGCAAGGTG-5′ | (SEQ ID NO:12) |
| insulin | 5′-GCCTTTGTGAACCAACACCTG-3′ | (SEQ ID NO:13) |
| | 3′-GTTGCAGTAGTTCTCCAGCTG-5′ | (SEQ ID NO:14) |
| glucagon | 5′-GAATTCATTGCTTGGCTGGT-3′ | (SEQ ID NO:15) |
| | 3′-CATTTCAAACATCCCACGTG-5′ | (SEQ ID NO:16) |
| somatostatin | 5′-CGTCAGTTTCTGCAGAAGTCC-3′ | (SEQ ID NO:17) |
| | 3′-CCATAGCCGGGTTTGAGTTA-5′ | (SEQ ID NO:18) |
| pancreatic polypeptide | 5′-GCTGCCGCACGCCTCTGCCTC-3′ | (SEQ ID NO:19) |
| | 3′-AAGTCCAGCGGGCTGAGCTCC-5′ | (SEQ ID NO:20) |
| amylase | 5′-CACTTTCCACCAGTCCCATA-3′ | (SEQ ID NO:21) |
| | 3′-GTTTACTTCCTCCAGGGAAAC-5′ | (SEQ ID NO:22) |

PCR was performed in Taq DNA polymerase buffer, containing 200 μm dNTP (Pharnacia), 0.5 mM MgCl2 (Sigma), 1 μM of each primer, and 2.5 U AmpliTaq DNA polymerase (Perkin-Elmer). PCR was carried out for 35 cycles in a Perkin-Elmer 2400 thermal cycler, with denaturing at 94° C., annealing at 55° C., and synthesis at 74° C.; incubations were for 45 s. Products were separated using a 1.5% agarose gel, and the gels were stained in 0.05% ethidium bromide.

### Example 5

TRAP Assay

Levels of telomerase activity were determined using the telomerase repeat amplification protocol (TRAP) assay, as described. Briefly, cells were solubilized in 160 μl of CHAPS lysis buffer and at least 1 ng of protein assayed. As negative controls, cell lysates were preincubated at 85° C. for 10 min to inactivate telomerase. Test samples were added to a reaction mixture of TRAP buffer, dNTP, 32P-end labeled TS primer (5′-AATCCGTCGAGCAGAGTT-3′ (SEQ ID NO: 23)), reverse primer (Intergen, Gaithersburg, Md.), T4 gene-32 protein (Boehringer Mannheim, Indianapolis, Ind.), and Taq polymerase (Promega). Following incubation for 30 min at 30° C. for telomerase-mediated extension of TS primer, the samples were then amplified by PCR, and the products resolved on a 12.5% non-denaturing PAGE gel, which was then analyzed by autoradiography.

### Example 6

Radioimmunoassay

A commercial immunoassay was used to measure human insulin levels in vitro, in both cells and supernatant. The cultured cells were first washed with PBS, and then fed with insulin-free basal media containing 25 mM (450 mg/dl) glucose. After three hours of high glucose exposure, both the supernatant and the pelleted cells were harvested and via RIA for both human insulin and insulin C-peptide, using the Linco Ultraspecific Human Insulin Assay Kit. To determine the total insulin synthesis for each culture, the values determined by RIA for both the cell pellets and supernatants were combined.

### Example 7

Nestin is Expressed by Undifferentiated Cells in the Fetal Human Pancreatic Islets

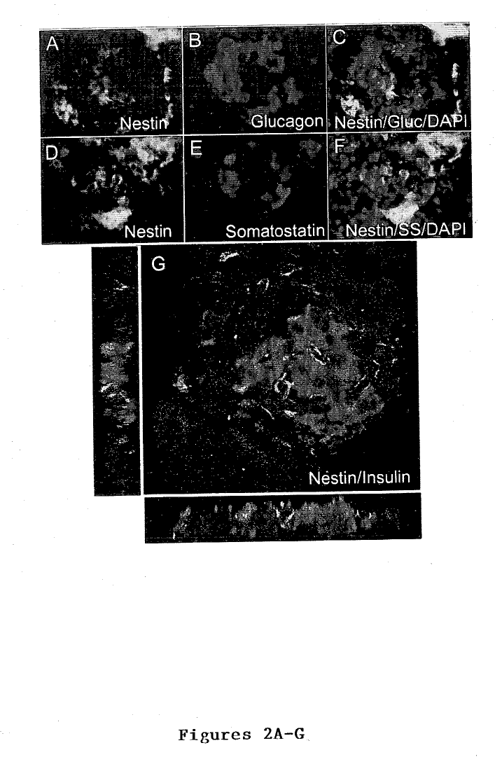

Nestin protein is expressed by cells in the pancreas, which have been postulated to be progenitor cells for either pancreatic acinar or endocrine lineages (Zulewski et al. “Multipotential Nestin-Positive Stem Cells Isolated From Adult Pancreatic Islets Differentiate ex vivo Into Pancreatic Endocrine, Exocrine, and Hepatic Phenotypes,” Diabetes 50(3):521-33 (2001), which is hereby incorporated by reference in its entirety). The question is whether the human fetal pancreatic primodium harbored nestin-expressing cells, and if so, whether these were progenitors for either the exocrine or endocrine lineages. To this end, second trimester pancreases of 16-23 weeks gestational age were immunolabeled for nestin, revealing that nestin protein was expressed both within nascent Islets (FIG. 2), and less so as single cells scattered throughout the basilar aspects of the developing acini, typically in a perivascular configuration. These perivascular nestin+ cells were epithelial in morphology, but expressed neither cytokeratin 19, a ductal marker (Franke et al. “Identification and Characterization of Epithelial Cells in Mammalian Tissues by Immunofluorescence Microscopy Using Antibodies to Prekeratin,” Differentiation 15(1):7-25 (1979); Bouwens et al. “Cytokeratins as Markers of Ductal Cell Differentiation and Islet Neogenesis in the Neonatal Rat Pancreas,” Diabetes 43(11):1279-83 (1994); Bouwens et al. “Identification of Rat Pancreatic Duct Cells by Their Expression of Cytokeratins 7, 19, and 20 in vivo and After Isolation and Culture,” J Histochem Cytochem 43(3):245-53 (1995); Gmyr et al. “Adult Human Cytokeratin 19-Positive Cells Reexpress Insulin Promoter Factor 1 in vitro: Further Evidence for Pluripotent Pancreatic Stem Cells in Humans,” Diabetes 49(10):1671-80 (2000), which are hereby incorporated by reference in their entirety), or amylase, an exocrine marker (Yasuda et al. “Localization by Immunofluroescence of Amylase, Trypsinogen and Chymotrypsinogen in the Acinar Cells of the Pig Pancreas,” J Histochem Cytochem 14(4):303-13 (1966), which is hereby incorporated by reference in its entirety). Similarly, the nestin+ cells found within presumptive Islets did not co-express any differentiated endocrine markers, such as glucagon, insulin, and somatostatin or pancreatic polypeptide (FIG. 2). Thus, in the late second trimester human fetal pancreas, nestin protein is expressed by relatively undifferentiated cells located in two distinct compartments, the presumptive Islets of Langerhans, and the perivascular space subjacent to the developing pancreatic acini.

### Example 8

The Nestin Enhancer Targeted GFP to Mitotic Pancreatic Progenitor Cells in Vitro

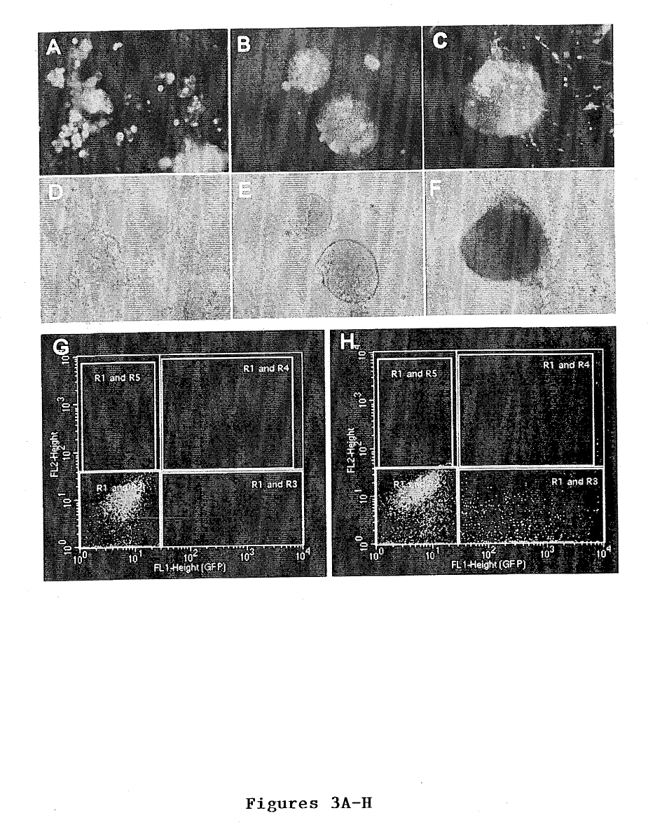

To identify live pancreatic progenitor cells, collagenase dissociates of 16-23 week gestational age (g.a.) pancreases were infected with adenoviruses bearing enhanced GFP (EGFP) under the regulatory control of the nestin enhancer (E/Nestin:EGFP), by an approach previously used to isolate neural stem cells from the developing forebrain (Keyoung et al. “High-Yield Selection and Extraction of Two Promoter-Defined Phenotypes of Neural Stem Cells From the Fetal Human Brain,” Nat Biotechnol 19(9):843-50 (2001), which is hereby incorporated by reference in its entirety. The dissociates were first prepared as suspension cultures, in DMEM/F12 supplemented with N2, and 10 ng/ml each of basic fibroblast growth factor (bFGF) and epidermal growth factor (EGF). The resultant cultures were then infected a day later with AdE/nestin:EGFP, over a range of 1-10 mol (multiplicity of infection/cell). Within four days in vitro, nestin-driven GFP expression was noted by cell clusters within the suspension cultures (FIG. 3). Among the E/nestin:EGFP+ cells, 98.1±1.0% expressed nestin protein (n=3 pancreases). Of these, 64±7.9% incorporated bromodeoxyuridine (BrdU) at 6 DIV, indicating their mitogenesis in vitro (FIG. 3). There was no co-labeling at 6 DIV with any endocrine markers, including insulin, glucagon, somatostatin, or pancreatic polypeptide. Thus, the nestin enhancer directed GFP expression to an initially undifferentiated population of mitotic cells in the human fetal pancreas.

### Example 9

E/nestin:GFP-Sorted Pancreatic Cells Expanded as Islet-Like Clusters in Vitro

After infection of the fetal pancreatic dissociates with AdE/nestin:EGFP virus, the nestin-driven GFP+ cells were isolated and extracted by FACS. By high-stringency FACS criteria intended for cell-type purification (Hunziker et al. “Nestin-Expressing Cells in the Pancreatic Islets of Langerhans,” Biochem Biophys Res Commun 271(1):116-9 (2000); Zulewski et al. “Multipotential Nestin-Positive Stem Cells Isolated From Adult Pancreatic Islets Differentiate ex vivo Into Pancreatic Endocrine, Exocrine, and Hepatic Phenotypes,” Diabetes 50(3):521-33 (2001), which are hereby incorporated by reference in their entirety), 4.0±0.8% of the cells (mean±SE; n=22 sorts) prepared from these 16-23 wk. g.a. dissociates were E/nestin:GFP+ (FIG. 3). When analyzed by gestational age, 2.1±0.5% of 20-23 week pancreatic cells were E/nestin:EGFP+(n=12), compared to 6.2±1.4% of 16-19 week pancreatic cells (n=10). The 16-19 week pancreases thus had a 3-fold higher proportion of E/nestin:GFP+ cells than their 20-23 week counterparts (p<0.01 by Student s t test), though in absolute terms, the larger 20-23 week pancreases harbored more total E/nestin:GFP+ cells.

FACS based on E/nestin-driven GFP thus permitted the isolation of a discrete pool of nestin-defined cells from the pancreatic primordium. Yet whereas 92.7±1.6% of AdE/nestin:EGFP-sorted cells expressed nestin protein immediately after FACS (n=3 sorts), only rare cells expressed insulin, glucagon, or somatostatin at that point (<1%). The undifferentiated state of these E/nestin.EGFP-sorted cells persisted for at least 14 DIV in minimal media.

To establish the lineage potential of E/nestin:EGFP-sorted pancreatic cells, low-density suspension cultures of E/nestin:EGFP sorted cells were next prepared as suspension cultures in DMEM/F12/N2 supplemented with 20 ng/ml each of EGF and bFGF, in order to allow the clonogenic expansion of any pancreatic progenitor cells therein (Schuldiner et al. “From the Cover: Effects of Eight Growth Factors on the Differentiation of Cells Derived From Human Embryonic Stem Cells,” Proc Natl Acad Sci USA 97(21):11307-12 (2000); Offield et al. “PDX-1 is Required for Pancreatic Outgrowth and Differentiation of the Rostral Duodenum,” Development 122(3):983-95 (1996), which are hereby incorporated by reference in their entirety). Under these culture conditions, it was observed the emergence of Islet-like clusters (ICC), that reliably arose during the two weeks after FACS (FIG. 3).

### Example 10

Activin Biases E/nestin-Sorted Islet Progenitors to Neuroendocrine Differentiation

To establish the neuroendocrine and beta cell potential of E/nestin:EGFP sorted pancreatic cells, the cells were next exposed to a variety of putative neuroendocrine differentiation agents, following mitogen withdrawal, and then assessed their antigenic phenotypes 7-9 days later. These agents, each assessed in a base medium of DMEM/F12/N2/10% FBS, included hepatocyte growth factor (HGF), whose secretion by fibroblasts has been shown to induce beta cell expansion (Otonkoski et al. “A Role for Hepatocyte Growth Factor/Scatter Factor in Fetal Mesenchyme-Induced Pancreatic Beta-Cell Growth,” Endocrinology 137(7):3131-9 (1996), which is hereby incorporated by reference in its entirety), nicotinamide, a nonspecific inducer of endocrine differentiation (Otonkoski et al. “Nicotinamide is a Potent Inducer of Endocrine Differentiation in Cultured Human Fetal Pancreatic Cells,” J Clin Invest 92(3): 1459-66 (1993), which is hereby incorporated by reference in its entirety), and activin A (4 nM), a TGFβ family member (Kessler et al. “Induction of Dorsal Mesoderm by Soluble, Mature Vg1 Protein,” Development 121(7):2155-64 (1995), which is hereby incorporated by reference in its entirety) that has been reported to inhibit endodermal sonic hedgehog signaling, and to thereby promote endocrine differentiation (Demeterco et al. “A Role for Activin A and Betacellulin in Human Fetal Pancreatic Cell Differentiation and Growth,” J Clin Endocrinol Metab 85(10):3892-7 (2000); Hebrok et al. “Notochord Repression of Endodermal Sonic Hedgehog Permits Pancreas Development,” Genes Dev 12(11): 1705-13 (1998); Hebrok et al. “Regulation of Pancreas Development by Hedgehog Signaling,” Development 127(22):4905-13 (2000), which are hereby incorporated by reference in their entirety). Each of these agents was added to E/nestin:GFP+ pancreatic cells immediately after their isolation via E/nestin:GFP-based FACS, as well as to unsorted control cultures. 7-9 days later, the cultured cells were immunolabeled to assess their expression of endocrine markers.

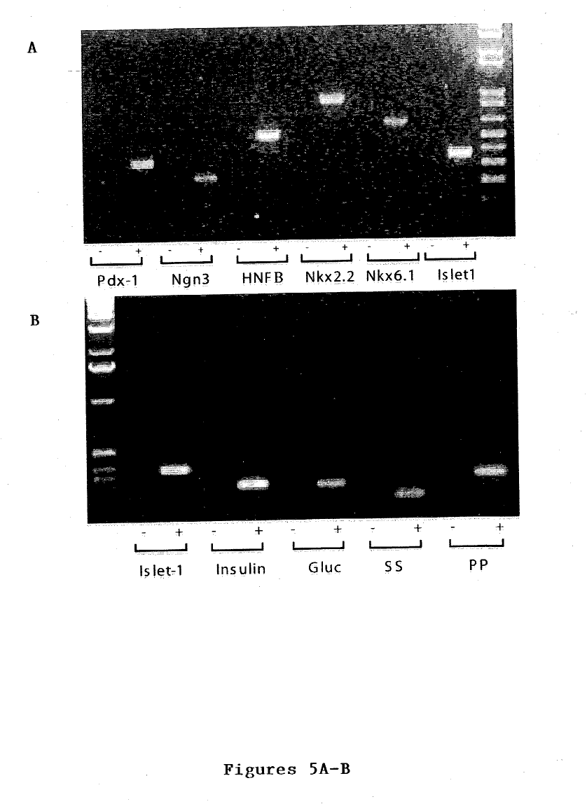

Among the treatment groups, only activin-A was associated with the development of pancreatic neuroendocrine cells, as defined by their expression of insulin, glucagon, somatostatin, or pancreatic polypeptide (FIG. 5). Thus, when E/nestin:GFP-sorted pancreatic cells were cultured in bFGF alone, only 1±0.7% (mean±SEM) became insulin+ at 14 DIV. Similarly, neither nicotinamide nor HGF induced endocrine differentiation by these cells (<1-2% for each). In contrast, 43.9±6.4 % of the E/nestin.EGFP+ cells exposed to activin-A for 9 days expressed insulin (n=854 cells, n=3 sorts). Moreover, most of the remaining cells expressed either glucagon, somatostatin, or pancreatic polypeptide. Thus, activin-A was associated with the development of pancreatic neuroendocrine cells (FIG. 5).

Interestingly, in unsorted cultures of pancreatic cells treated with activin A, 9.2±2% developed insulin-IR (n=1366 cells, n=3 samples). Thus, sorting based on E/nestin:GFP resulted in a 4.8-fold increase in the fraction of activin-induced beta cells, relative to the unsorted parental cultures from which they were derived (F=81.247 [1, 3 d.f.]; p<0.01, by ANOVA with Boneferroni post hoc comparisons). Together, these data indicated that the fraction of insulin+ cells in these pancreatic cultures rose from 2.9% in otherwise untreated pancreatic dissociates after 14 DIV, to 44% in cultures of E/nestin.EGFP-sorted cells exposed thereafter to activin A (FIG. 5). In relative terms then, E/nestin.EGFP-based isolation performed in tandem with activin exposure yielded a net 15-fold enrichment of insulin producing cells, compared to native unsorted cells raised under basal bFGF-conditions.

### Example 11

Both α and δ Islet Cells were also Generated from E/nestin:EGFP-Sorted Cells

Activin-treatment of E/nestin:GFP-sorted pancreas was similarly associated with the selective generation of Islet alpha and delta cells. By 9 DIV, 41.1±4.7% of the cells arising from E/nestin.EGFP+ progenitors (n=840 cells, n=3 sorts) expressed glucagon, compared to 16.3±2.1 % of their unsorted controls (n=576 cells, n=3 samples), a 2.5 fold enrichment (FIG. 5). Similarly, 43±17% expressed somatostatin, vs. 11±1.4% in unsorted cells. Although the total percentage of endocrine cells exceeded 100 percent, transient co-expression of glucagon and somatostatin, as has been reported during normal development (Teitelman et al., “Precursor Cells of Mouse Endocrine Pancreas Co-Express Insulin, Glucagons and the Neuronal Proteins Tyrosine Hydroxylase and Neuropeptide Y but not Pancreatic Polypeptide,” Development 118(4):1031-39 (1993); Larson, “On the Development of the Islets of Langerhans,” Micro. Res. Tech. 43:284-91 (1998), which are hereby incorporated by reference in their entirety), was often noted. The vast majority of cells in these cultures expressed one or the other of the three endocrine markers. In addition, it was found that at 9 DIV, 24±8.1% expressed pancreatic polypeptide. In contrast, 8.6%±2.0 % of unsorted cells differentiated to pancreatic polypeptide-expressing cells.

On the basis of these observations, it was concluded that E/nestin-based FACS significantly enriched a population of Islet progenitor cells, that were competent to give rise to all major pancreatic neuroendocrine phenotypes. Activin-A specifically promoted this process, by strongly biasing E/nestin:GFP-sorted pancreatic progenitors to alpha and beta cell, and delta cell maturation. Activin had substantially less effect in unsorted controls, arguing that E/nestin:EGFP selectively enriched an Islet progenitor pool competent to respond to activin with neuroendocrine differentiation.

### Example 12

E/nestin-Sorted Cells may be Expanded as Committed Endocrine Progenitors

To assess the endocrine commitment of cells derived from E/nestin:GFP-sorted progenitors, Islet-like clusters derived from E/nestin-sorted 16 wk pancreas were redissociated to low-density culture, at 10⁴ cells/ml. This allowed assessment of the clonal derivatives of single propagated Islets, and hence the phenotypic range of single Islet progenitors. The progeny of the resultant secondary and tertiary ICC were plated onto Matrigel and allowed to differentiate, and then immunolabeled to establish their phenotypes. They were found to gave arise to endocrine cells that expressed insulin, glucagon, somatostatin, and pancreatic polypeptide as well. Thus, E/nestin:EGFP-sorted cells continued to divide in vitro, and gave rise individually to all endocrine cell types. Of note, when raised on Matrigel and in the absence of activin A, Islet-like clusters also gave rise to a proportion of exocrine cells, as defined by their expression of amylase. This observation argues that activin's role in supporting neuroendocrine differentiation from E/nestin:GFP-sorted pancreatic cells may be specifically instructive, and that the E/nestin:GFP-defined pancreatic progenitor may have lineage competence for exocrine as well as Islet derivatives.

### Example 13

AdE/nestin:EGFP-Sorted Cells Generated Islet-Like Cell Clusters

To assess the incidence of clonogenic progenitors within the e/nestin:GFP-sorted pool, limiting dilution analysis was performed on AdE/nestin:EGFP-sorted cells, as well as on matched populations of AdE/nestin:EGFP-depleted cells and unsorted cells. The sorted cells were plated at densities of 1, 10, 100, 1000, and 5000 cells in 96 well plates at 200 μl/well, in culturing medium with 10 ng/ml each of bFGF and EGF. The cells were followed for two weeks and the number of ICC-generated were quantified. A density-dependent generation of ICC was observed, such that clusters appeared in E/nestin:GFP-sorted cultures only at densities of >100,000 cells/ml, at which 30 ICC clusters were generated per 100,000 cells.

### Example 14

E/nestin:GFP-Sorted Pancreatic Cells Retain Telomerase Activity

To determine the mitotic potential of E/nestin:EGFP-sorted Islet progenitors, telomerase activity was assessed using the telomeric repeat amplification protocol (TRAP). The TRAP assay defines the extent to which cells possess telomerase enzymatic activity, which typically is measurable only in stem cells, early development, and in tumors. Immediately after FACS of E/nestin:GFP-sorted pancreatic cells, the cells were lysed and the TRAP assay performed. Whereas as few as 5,000 nestin-sorted cells exhibited readily detectable telomerase activity, quantities of up to 10,000 nestin-depleted pancreatic cells did not (FIG. 5). The expression of telomerase activity by E/nestin-defined cells suggests their marked proliferative potential, and further argues that the E/nestin:GFP-defined pool includes pancreatic stem cells.

### Example 15

E/nestin:GFP-Isolated Progenitors Exhibit an Early Islet Transcription Factor Profile

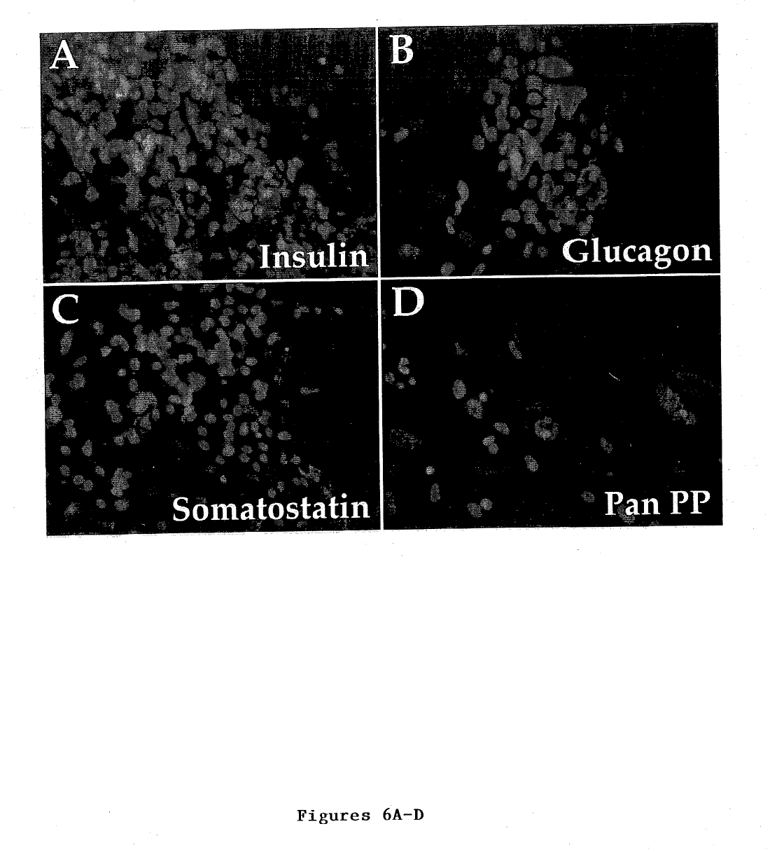

To assess the transcription factor profile of E/nestin:EGFP-sorted putative pancreatic progenitors, RT-PCR was used to amplify phenotype-selective transcripts from AdE/nestin:EGFP-sorted human pancreatic cells, which underwent RNA extraction immediately upon FACS. Prior to FACS, these cells had been cultured in a basal media of DMEM/F12/N2 supplemented only with bFGF and EGF. Immediately upon FACS, the extracted pool of E/nestin:EGFP+ cells was noted to express the endodermal transcription factor HNF-3β, the pancreatic homeodomain transcription factor PDX-1 (Ahlgren et al. “The Morphogenesis of the Pancreatic Mesenchyme is Uncoupled From That of the Pancreatic Epithelium in IPF1/PDX1-Deficient Mice,” Development 122(5): 1409-16 (1996), which is hereby incorporated by reference in its entirety), which is HNF-3b regulated (Wu et al. “Hepatocyte Nuclear Factor 3beta is Involved in Pancreatic Beta-Cell-Specific Transcription of the pdx-1 Gene,” Mol Cell Biol 17(10):6002-13 (1997), which is hereby incorporated by reference in its entirety), as well as the early neurogenic transcription factors Nkx6.1, Nkx2.2, Islet-1, and Ngn3 (FIG. 6A). The latter is of particular note, since ngn3 appears to be expressed specifically by endocrine progenitor cells, whereas pdx-1 is expressed by both endocrine and exocrine pancreas, and is required for pancreatic differentiation (Jonsson et al. “Insulin-Promoter-Factor 1 is Required for Pancreas Development in Mice,” Nature 371(6498):606-9 (1994); Offield et al. “PDX-1 is Required for Pancreatic Outgrowth and Differentiation of the Rostral Duodenum,” Development 122(3):983-95 (1996); which are hereby incorporated by reference in their entirety). Upon exposure to 10% FBS, the AdE/Nestin/EGFP+ cells quickly differentiated to additionally express Nkx6.1, Islet-1, somatostatin, pancreatic polypeptide, insulin, and glucagon, indicating their differentiation as neuroendocrine Islet cells. Thus, E/nestin-defined fetal pancreatic cells express a transcription factor profile (hnf-3β, pdx-1) typical of undifferentiated pancreatic progenitors, and differentiate to express transcription factors (nkx2.2, nkx6.1, ngn3, and Islet 1) and hormones (SS, PP, insulin, and glucagon) typical of neuroendocrine cells. These findings indicate that fetal human pancreatic cells recognized by E/nestin-driven EGFP include Islet progenitor cells.

### Example 16

Beta Cells Derived from Sorted Human Islet Progenitor Cells Regulated Insulin in Response to High-Glucose

To determine the ability of human Islet progenitor-derived beta cells to secrete insulin in response to glucose fluctuation, RIA was used to measure the levels of insulin secreted into the media by Activin A-differentiated cells, after a 3 hr challenge with 25 mM glucose. To this end, human-specific insulin radioimmunoassay (RIA) was used to assess their secretion of insulin in response to high glucose (25 mM, or 450 mg/dl). E/nestin:EGFP-based sorting was first used to enrich neuroendocrine progenitors from fetal human pancreatic dissociates. These were then raised in activin A and betacellulin for 9 days to trigger their insulinergic differentiation (Mashima et al., “Betacellulin and Activin A Coordinately Convert Amylase-Secreting Pancreatic AR42J Cells into Insulin-Secreting Cells,” J. Clin. Invest. 97(7):1647-54 (1996) and Demeterco et al. “A Role for Activin A and Betacellulin in Human Fetal Pancreatic Cell Differentiation and Growth,” J Clin Endocrinol Metab 85(10):3892-7 (2000), which are hereby incorporated by reference in their entirety). After three hours of high glucose exposure, the E/nestin:EGFP-sorted cells generated 53.9±13 ng insulin/μg protein (n=7 samples). In contrast, unsorted cultures generated 13.6±2.3 ng insulin/μg protein (n-7), while E/nestin:EGFP-depleted cultures had 13.1±3.9 ng insulin/μg (n=4) (Table 2).

TABLE 2

| | Nestin-sorted | Nestin:EGFP | Unsorted |
|---|---|---|---|
| Normal Islet Cell: | Positive cells | Depleted cells | cells |
| (50 ng of Insulin | (ng Insulin/ | (ng Insulin/ | (ng Insulin/ |
| per μg of Protein) | μg of Protein) | μg of Protein) | μg of Protein) |
| Cell Extract | 53.9 ± 13 | 13.1 ± 3.9 | 13.6 ± 2.3 |
| Insulin Production | | | |

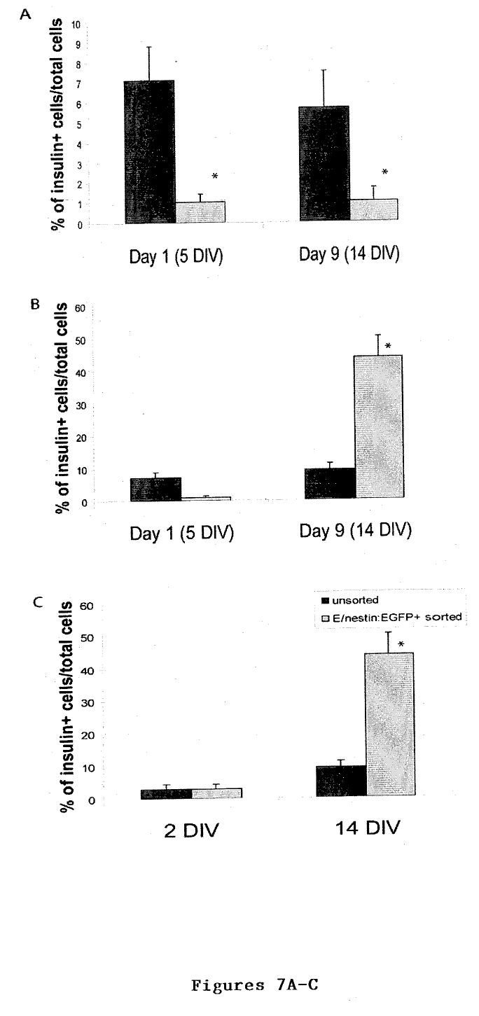

Thus, AdE/nestin:EGFP-sorted Islet progenitors gave rise to beta cell daughters able to respond to glucose with over a 4-fold increase in insulin production, compared to their unselected or negatively selected controls (p<0.05 by one-way ANOVA with Tukey/Kramer post hoc testing; F=5.01; [2, 15 d.f ]) (FIG. 7). The E/nestin.EGFP-sorted cells, raised serially in bFGF and activin for 9 days after FACS, responded to glucose challenge by secreting 615±41 pg insulin/μg of protein (n=4). This represented a sharp increase relative to their unstimulated E/nestin:GFP-sorted controls, whose supernatants achieved insulin levels of only 250μ23 pg/μg of protein (FIG. 7). By way of comparison, unsorted cells otherwise raised identically responded to glucose challenge by secreting 431±25 pg (n=4) of insulin per μg of protein, compared to their basal level of 257±69 pg insulin/pg protein after 2 days in culture (Table 3).

TABLE 3

| Insulin Secretion | | Post-Glucose |
|---|---|---|
| (ng Insulin secreted per μg | | Challenge |
| of protein) | Basal | (25 mM) |
| Nestin + Cells | 282 | 615 |
| Nestin Negative Cells | 250 | 384 |
| Unsorted Cells | 257 | 431 |

Those cells derived from nestin-negative cells secreted 384±31 pg insulin/μg protein (n=4) after glucose challenge, up from their unstimulated level of 282±33 pg/μg protein. Thus, cells derived from E/nestin:GFP-sorted progenitors secrete insulin in a glucose-responsive manner, preferentially so with regards to both their unsorted and negatively selected controls.

In this study, nestin-specified GFP-based fluorescence-activated cell sorting (FACS) was used to identify and isolate Islet progenitor cells from the human fetal pancreas. This was accomplished using both plasmid and adenoviral expression vectors to insert transgenes encoding GFP, placed under the regulatory control of the nestin enhancer, into acute dissociates of the human pancreas. The progenitors thus identified were extracted by FACS, and were found to be mitotically active and readily expandable in FGF2. They generated morphologically Islet-like clusters in vitro, which indeed assumed Islet phenotype, as defined by neural markers that included synaptophysin and nestin protein, and Islet cell markers that included Islet-1 and PDX-1. Importantly, the nestin-defined Islet progenitor cells were found to differentiate into exclusively neuroendocrine progeny upon activin exposure in vitro, yielding substantially pure populations of alpha, beta, and delta Islet cells. Interestingly, no E/nestin:EGFP co-expression was observed with any of the differentiated endocrine markers, suggesting that nestin expression characterized Islet progenitors, but not their progeny. Nonetheless, once exposed to activin so as to induce their neuroendocrine differentiation, the E/nestin:EGFP-sorted cells rapidly matured as neuroendocrine cells, almost half as beta cells that synthesized and secreted insulin in response to glucose challenge.

Insulin Production/Cell Therapy

Prior studies of progenitor-derived insulin-secreting cells have included both tissue and embryonic stem-cell derived beta cells. Progress towards generating cultured populations of insulin-secreting cells has been particularly notable using ES cells, where great hope has attached to the in vitro generation of ES-derived beta cells, and the persistent production of insulin by these cells, both in vivo and in vitro. However, the quantitative levels of insulin production achieved by these cells has thus far been disappointing, <3 ng of insulin/mg protein within 5 min of a 20 mM glucose stimulation. In contrast, it has been noted here that nestin-sorted human Islet progenitors, raised in activin for 9 div, produce >50 ng insulin/μg protein in response to 25 mM glucose challenge after 3 hours. This is within the range of both rodent Islet cells, which may synthesize 50-500 ng insulin/mg protein (Zulewski et al. “Multipotential Nestin-Positive Stem Cells Isolated From Adult Pancreatic Islets Differentiate ex vivo Into Pancreatic Endocrine, Exocrine, and Hepatic Phenotypes,” Diabetes 50(3):521-33 (2001), which is hereby incorporated by reference in its entirety), and adult human cadaveric Islet cells which secrete 200 ng Insulin/ug protein per hour (Lukowiak et al., “Indentification and Purification of Functional Human Beta-Cells by a New Specific Zinc-Fluorescent Probe,” J. Histochem. Cytochem. 49:519-28 (2001), which is hereby incorporated by reference in its entirety).

Islet Progenitors may have Broader Lineage Competence

Upon initial isolation, the nestin-sorted pancreatic cells expressed transcription factors indicative of early endodermal and pancreatic exocrine phenotype, as well as transcripts more suggestive of neuroendocrine lineage. Their early expression of ngn3 is of particular note, since ngn3 may be expressed specifically by endocrine progenitor cells. In contrast, pdx-1 is expressed by both endocrine and exocrine pancreas, and is required for pancreatic differentiation (Jonsson et al. “Insulin-Promoter-Factor 1 is Required for Pancreas Development in Mice,” Nature 371(6498):606-9 (1994); Offield et al. “PDX-1 is Required for Pancreatic Outgrowth and Differentiation of the Rostral Duodenum,” Development 122(3):983-95 (1996), which are hereby incorporated by reference in its entirety). It is possible that this reflects a heterogeneous progenitor pool, comprised of both endodermal and neural parental progenitors. Alternatively, it is possible that the E/nestin-defined fetal human progenitor cells are of endodermal origin, with secondary neuroendocrine differentiation from endodermal progenitors. The latter possibility is suggested by the additional observation that amylase, a pancreatic exocrine marker, is also expressed by a subpopulation of daughters derived from E/nestin-sorted cells, at least when raised in the absence of activin. At this point, the above data does not allow one to distinguish between these possibilities, absent formal lineage analysis.

Islet Progenitors may be Serially Isolated and Induced to Beta Cell Differentiation

Any ambiguity regarding the lineage potential of E/nestin:GFP pancreatic progenitors was rendered moot by their almost uniform differentiation into neuroendocrine cells after differentiation in activin and betacellulin. The predominantly neuroendocrine fate of these cells upon differentiation indicates that E/nestin-driven EGFP can be used to identify and extract Islet progenitors from the developing pancreas. Islet progenitors may thereby now be prospectively identified, expanded and enriched in a form that permits their activin-triggered neuroendocrine differentiation, with the high-efficiency generation of α, β, and δ cells. As such, this approach may permit the selective isolation of Islet progenitors from human fetal tissues, and their directed differentiation to insulin-secreting β cells, in numbers and purity appropriate for allograft treatment of diabetes mellitus.

Although preferred embodiments have been depicted and described in detail herein, it will be apparent to those skilled in the relevant art that various modifications, additions, substitutions and the like can be made without departing from the spirit of the invention and these are therefore considered to be within the scope of the invention as defined in the claims which follow.

What is claimed:

1. A method of separating pancreatic Islet cells or progenitor or stem cells thereof from a mixed population of cells from the pancreas comprising:
selecting an enhancer/promoter which functions in said pancreatic Islet cells or progenitor cells thereof;
introducing a nucleic acid molecule encoding a fluorescent protein under control of said enhancer/promoter into the mixed population of cells;
allowing the pancreatic Islet cells or progenitor or stem cells thereof to express the fluorescent protein; and
separating the fluorescent cells from the mixed population of cells, wherein said separated cells are said pancreatic Islet cells or progenitor or stem cells thereof.

2. The method of claim 1, wherein said introducing comprises viral mediated transduction of said pancreatic Islet stem cells and progenitors thereof.

3. The method of claim 2, wherein said viral mediated transduction comprises adenovirus-mediated transduction.

4. The method of claim 2, wherein said viral mediated transduction comprises retroviral-mediated transduction.

5. The method of claim 2, wherein said viral mediated transduction comprises lentiviral-mediated transduction.

6. The method of claim 2, wherein said viral mediated transduction comprises transduction by adeno-associated virus.

7. The method of claim 1, wherein said introducing comprises electroporation.

8. The method of claim 1, wherein said introducing comprises naked DNA insertion.

9. The method of claim 8, wherein said introducing comprises biolistic penetration.

10. The method of claim 1, wherein said introducing comprises liposomal-mediated transformation of said mixed population of cells.

11. The method of claim 1, wherein said separating comprises fluorescence activated cell sorting.

12. The method of claim 1, wherein said enhancer/promoter is an E/nestin enhancer/promoter.

13. The method of claim 1, wherein said enhancer/promoter is a Musashi promoter.

14. The method of claim 1, wherein said enhancer/promoter is an NKX6.1 promoter.

15. The method of claim 1, wherein said enhancer/promoter is a neurogenin-3 promoter.

16. The method of claim 1, wherein said enhancer/promoter is an HB9 promoter.

17. The method of claim 1, wherein said enhancer/promoter is an PDX-1 promoter.

18. The method of claim 1, wherein the mixed population of cells is derived from pancreatic tissue.

19. The method of claim 1, wherein the mixed population of cells is derived from pancreatic cell culture.

20. The method of claim 1, wherein the pancreatic Islet cells or progenitor or stem cells thereof are human.

21. The method of claim 15, wherein the pancreatic Islet cells or progenitor or stem cells thereof are of adult origin.

22. The method of claim 15, wherein the pancreatic Islet cells or progenitor or stem cells thereof are of fetal origin.

23. An enriched or purified preparation of isolated human pancreatic Islet cells or progenitor or stem cells.

24. The enriched or purified preparation of claim 23, wherein the cells are of adult origin.

25. The enriched or purified preparation of claim 23, wherein the cells are of fetal origin.

26. A method of treating a diabetic condition comprising:
providing the enriched or purified preparation of pancreatic Islet progenitor or stem cells or Islet cells differentiated therefrom and
transplanting the cells into a subject under conditions effective to treat a diabetic condition.

27. A method of treating a diabetic condition according to claim 26, wherein the diabetic condition is Diabetes Mellitus Type 1.

28. A method of treating a diabetic condition according to claim 26, wherein the diabetic condition is Diabetes Mellitus Type 2.

29. A method of treating a diabetic condition according to claim 26, wherein the cells are human.

30. A method of treating a diabetic condition according to claim 29, wherein the cells are of adult origin.

31. A method of treating a diabetic condition according to claim 29, wherein the cells are of fetal origin.

32. A method of treating a diabetic condition according to claim 29, wherein the cells produce insulin.

33. A method of treating a diabetic condition according to claim 29, wherein the cells produce glucagon.
